(19) United States
(12) Patent Application Publication
(10) Pub. No.: US 2025/0375145 A1
(43) Pub. Date: Dec. 11, 2025

(54) NEURAL MULTIFUNCTIONAL DEVICE AND METHOD OF MANUFACTURING THE SAME

(71) Applicant: SEOUL NATIONAL UNIVERSITY R&DB FOUNDATION, Seoul (KR)

(72) Inventors: Gyuchul Yi, Seoul (KR); Jamin Lee, Seoul (KR)

(21) Appl. No.: 18/686,484

(22) PCT Filed: Mar. 11, 2022
(86) PCT No.: PCT/KR2022/003431

§ 371 Date: Jan. 28, 2025

(30) Foreign Application Priority Data
Sep. 2, 2021 (KR) 10-2021-0117242

(51) Int. Cl. A61B 5/388; A61B 5/00; A61B 5/294; A61N 1/36

(52) U.S. Cl. CPC A61B 5/388; A61B 5/294; A61B 5/4839; A61N 1/3605; A61B 2562/0285; A61B 2562/12

(57) ABSTRACT

The present disclosure provides a neural manufacturing device includes a two-dimensional nanomaterial layer in which a plurality of holes are formed, a mask layer disposed on the two-dimensional nanomaterial layer and having a plurality of openings each exposing the plurality of holes, a plurality of nanotubes each vertically disposed on the region of the two-dimensional nanomaterial layer exposed around the plurality of holes by the mask layer and having a passage for substance movement therein, a binding layer disposed on the mask layer to fill a region between and around the plurality of nanotubes to a given height, and an electrode structure disposed on the binding layer to be electrically connected to at least a portion of the plurality of nanotubes.

### TECHNICAL FIELD

The present invention relates to a device related to a nerve cell and a manufacturing method thereof, and more specifically, to a device capable of performing electrical measurement on a nerve cell and a manufacturing method thereof.

### BACKGROUND ART

Existing signal measurement devices for nerve cells use needle-type probes using nanowires, so they have the limitation of only being able to measure signals. Therefore, in order to supply a nerve substance (medicinal substance) into a nerve cell during signal measurement using a signal measurement device, a separate nerve substance delivery device (passage) must be provided. However, in this case, it is not only easy to place the nerve substance delivery device (passage) by avoiding the signal measurement device, but the accuracy of nerve substance transfer may also be reduced. Furthermore, because two devices must be handled, the measurement process may be complicated and difficult.

In addition, because existing signal measurement devices for nerve cells generally have a rigid device form, they have the disadvantage of being quite limited in their utilization and application fields.

### DISCLOSURE OF THE INVENTION

### Technical Problem

The present disclosure is directed to providing an electrolyte for a lithium metal battery that can prevent dendrite by forming a LiF-rich solid-electrolyte interphase on the surface of a lithium metal anode.

The present disclosure is also directed to providing a lithium metal battery with superior high voltage and cycle characteristics and improved stability, which includes the electrolyte for a lithium metal battery.

### Technical Solution

The technological object to be achieved by the present invention is to provide a neural multifunctional device which may perform at least one operation of electrical measurement and stimulation application to a nerve cell and also perform delivery of a substance (medicinal substance) to a nerve cell.

In addition, the technological object to be achieved by the present invention is to provide a neural multifunctional device which may be easily implemented as a form of a flexible device by using a two-dimensional nanomaterial.

In addition, the technological object to be achieved by the present invention is to provide a manufacturing method of the above-described neural multifunctional device.

The objects to be achieved by the present invention is not limited to the objects mentioned above, and other objects not mentioned will be understood by those skilled in the art from the description below.

According to one embodiment of the present invention, there is provided a neural multifunctional device comprising: a two-dimensional nanomaterial layer in which a plurality of holes are formed; a mask layer disposed on the two-dimensional nanomaterial layer and having a plurality of openings each exposing the plurality of holes, wherein the opening has a larger diameter than that of the hole corresponding thereto and is formed to expose a region of the two-dimensional nanomaterial layer around the hole; a plurality of nanotubes each vertically disposed on the region of the two-dimensional nanomaterial layer exposed around the plurality of holes by the mask layer and having a passage for substance movement therein; a binding layer disposed on the mask layer to fill a region between and around the plurality of nanotubes to a given height; and an electrode structure disposed on the binding layer to be electrically connected to at least a portion of the plurality of nanotubes, and wherein the plurality of nanotubes are configured to perform at least one of electrical measurement and stimulation application to a nerve cell and transfer of substance to the nerve cell through the passage.

The two-dimensional nanomaterial layer may include graphene.

The plurality of nanotubes may be formed of a metal oxide.

The plurality of nanotubes may be formed of a Zn oxide.

Each of the plurality of nanotubes may have an inner diameter ranging from about 220 nm to 2 μm and an outer diameter ranging from about 330 nm to 2.5 μm.

Each of the plurality of nanotubes may have a length ranging from about 600 nm to 12 μm.

The plurality of nanotubes may have a protruding shape with respect to a surface of the binding layer.

The binding layer may include polymer.

The neural multifunctional device may be a flexible device.

According to another embodiment of the present invention, there is provided a manufacturing method of neural multifunctional device comprising: forming a stack in which a two-dimensional nanomaterial layer and a mask layer are sequentially arranged on a substrate, wherein the two-dimensional nanomaterial layer has a plurality of holes, the mask layer has a plurality of openings each exposing the plurality of holes, and the opening has a larger diameter than that of the hole corresponding thereto and exposes a region of the two-dimensional nanomaterial layer around the hole; forming a plurality of nanotubes each vertically disposed on the region of the two-dimensional nanomaterial layer exposed around the plurality of holes by the mask layer and having a passage for substance movement therein; forming a binding layer on the mask layer to fill a region between and around the plurality of nanotubes to a given height; and forming an electrode structure arranged to be electrically connected to at least a portion of the plurality of nanotubes on the binding layer, and wherein the plurality of nanotubes are configured to perform at least one of electrical measurement and stimulation application to a nerve cell and transfer of substance to the nerve cell through the passage.

After the forming the binding layer or the forming the electrode structure, the manufacturing method may further include separating a device structure including at least the two-dimensional nanomaterial layer, the mask layer, the plurality of nanotubes, and the binding layer from the substrate.

The two-dimensional nanomaterial layer may include graphene.

The plurality of nanotubes may be formed of a metal oxide.

The plurality of nanotubes may be formed of a Zn oxide.

The plurality of nanotubes may have a protruding shape with respect to a surface of the binding layer.

### ADVANTAGEOUS EFFECTS

According to embodiments of the present invention, it is possible to implement a neural multifunctional device which may perform at least one of electrical measurement and stimulation application to a nerve cell and also perform delivery of a substance (medicinal substance) to a nerve cell. Since electrical measurement and/or stimulation and substance transfer for a nerve cell may be performed together by using a single device, the effects that it is easier to make measurements and measurement accuracy is improved may be obtained. In particular, since a certain substance (medicinal substance) may be delivered to nerve cells and the response thereto may be easily measured electrically, research or experiments on nerve cells may be facilitated, and evaluation of a certain medicinal substance may also be facilitated.

In addition, according to embodiments of the present invention, as it is possible to implement a neural multifunctional device which may be easily manufactured as a form of a flexible device by using a two-dimensional nanomaterial, it may be advantageous in expanding the utilization and application fields of the above-mentioned neural multifunctional devices.

### BRIEF DESCRIPTION OF THE DRAWINGS

FIG. 1 is a cross-sectional diagram illustrating a neural multifunctional device according to an embodiment of the present invention.

FIG. 2 is a schematic diagram illustrating a process for performing electrical measurement and substance transfer to a nerve cell using a neural multifunctional device according to an embodiment of the present invention.

FIG. 3A to FIG. 3F are cross-sectional diagrams illustrating a manufacturing method of a neural multifunctional device according to an embodiment of the present invention.

FIG. 4 is a perspective diagram illustrating the structure of FIG. 3A in three dimensions.

FIG. 5 is a perspective diagram illustrating the structure of FIG. 3B in three dimensions.

FIG. 6 is a perspective diagram illustrating the structure of FIG. 3C in three dimensions.

FIG. 7A to FIG. 7C are electron microscope images of a plurality of nanotubes formed to have different inner diameters, outer diameters, and heights according to an embodiment of the present invention.

FIG. 8 is an electron microscope image of a portion of a neural multifunctional device manufactured according to an embodiment of the present invention.

FIG. 9 is an electron microscope image taken from above of a portion of a neural multifunctional device manufactured according to an embodiment of the present invention.

FIG. 10 is an electron microscope image showing a case where nerve cells were cultured on a neural multifunctional device manufactured according to an embodiment of the present invention.

### BEST MODE FOR CARRYING OUT THE INVENTION

Hereinafter, embodiments of the present invention will be described in detail with reference to the accompanying drawings.

The embodiments of the present invention to be described below are provided to more clearly explain the present invention to those skilled in the art, and the scope of the present invention is not limited by the following embodiments, and the embodiments may be modified in many different forms.

The terms used in this specification are used to describe specific embodiments and are not intended to limit the present invention. The terms indicating a singular form used herein may include plural forms unless the context clearly indicates otherwise. Also, as used herein, the terms, “comprise” and/or “comprising” specify the presence of the stated shape, step, number, operation, member, element, and/or group thereof and does not exclude the presence or addition of one or more other shapes, steps, numbers, operations, elements, elements and/or groups thereof. In addition, the term, “connection” used in this specification means not only a direct connection of certain members, but also a concept including an indirect connection in which other members are interposed between the members.

In addition, in the present specification, when a member is said to be located “on” another member, this arrangement includes not only a case in which a member is in contact with another member, but also a case where another member exists between the two members. As used herein, the term, “and/or” includes any one and all combinations of one or more of the listed items. In addition, the terms of degree such as “about” and “substantially” used in the present specification are used as a range of values or degrees, or as a meaning close thereto, taking into account inherent manufacturing and substance tolerances, and exact or absolute figures provided to aid in the understanding of this application are used to prevent the infringers from unfairly exploiting the stated disclosure.

Hereinafter, embodiments of the present invention will be described in detail with reference to the accompanying drawings. A size or a thickness of areas or parts shown in the accompanying drawings may be slightly exaggerated for clarity of the specification and convenience of description. The same reference numbers indicate the same configuring elements throughout the detailed description.

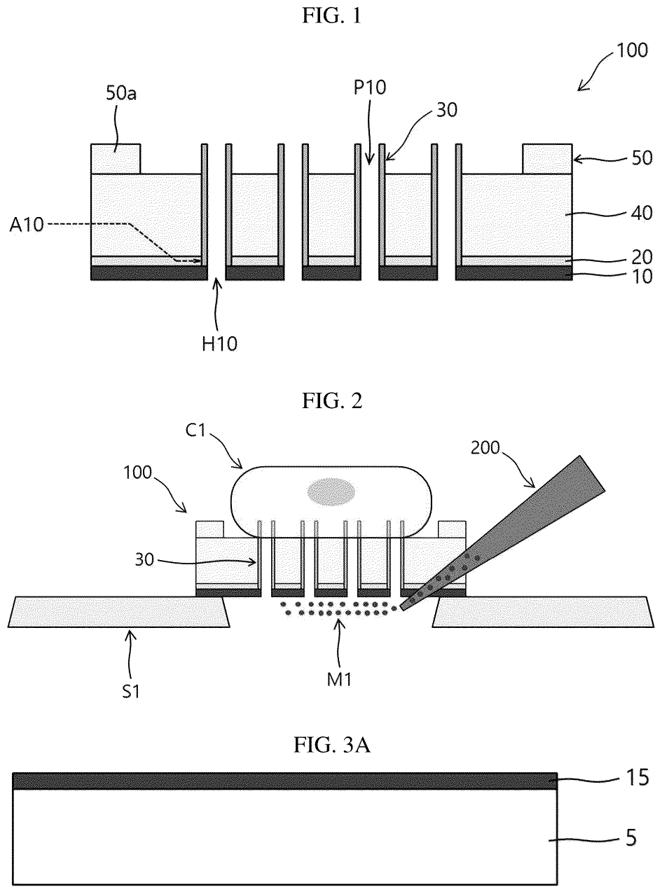

FIG. 1 is a cross-sectional diagram illustrating a neural multifunctional device 100 according to an embodiment of the present invention.

Referring to FIG. 1, the neural multifunctional device 100 according to an embodiment of the present invention may include a two-dimensional nanomaterial layer 10. The two-dimensional nanomaterial layer 10 may include, for example, graphene. In one embodiment, the two-dimensional nanomaterial layer 10 may be composed of a graphene layer. A two-dimensional material (2D material) may be a single-layer, half-layer, or two-to three-layer layered structure in which atoms form a predetermined crystal structure. Graphene is a two-dimensional material, and a single-layer (monoatomic layer) structure in which carbon atoms form a hexagonal structure. Graphene may have a symmetrical band structure based on the Dirac point, and because the effective mass of charge at the Dirac point is very small, it may have charge mobility which is at least 10 times faster (up to 1,000 times more) than silicon (Si). Furthermore, graphene may have a very large Fermi velocity (V_{F}). Graphene may be an excellent conductor and a flexible nanomaterial. In an embodiment of the present invention, the two-dimensional nanomaterial layer 10 may include a single-layer or a multi-layer graphene. Electro-structurally, a two-dimensional material may be defined as a material whose density of state (DOS) follows quantum well behavior. Since the density of state (DOS) may follow quantum well behavior even in a material in which a plurality of two-dimensional unit material layers are stacked (up to about 100 layers or up to about 20 layers), a structure in which the two-dimensional unit material layers (e.g., single graphene) are repeatedly stacked may also be called as a ‘two-dimensional material’ from this perspective. The two-dimensional nanomaterial layer 10 may be said to have a two-dimensional layered structure. The two-dimensional nanomaterial layer 10 may function as a kind of ‘a common electrode’.

In an embodiment of the present invention, a plurality of holes H10 may be formed in the two-dimensional nanomaterial layer 10. The plurality of holes H10 may be formed to penetrate through the two-dimensional nanomaterial layer 10 in the thickness direction, and may be arranged regularly or substantially regularly.

The neural multifunctional device 100 may include a mask layer 20 disposed on the two-dimensional nanomaterial layer 10. The mask layer 20 may have a plurality of openings A10 each exposing the plurality of holes H10 of the two-dimensional nanomaterial layer 10. The opening A10 may have a larger diameter than that of the hole H10 corresponding thereto. For example, the opening A10 and the corresponding hole H10 may have the same center, and the diameter of the opening A10 may be larger than the diameter of the hole H10. Accordingly, a region of the two-dimensional nanomaterial layer 10 around the hole H10 may be exposed by the opening A10. The region of the two-dimensional nanomaterial layer 10 exposed around the hole H10 may have a ring shape when observed from above.

The mask layer 20 may be formed of a certain insulating material. For example, the mask layer 20 may be formed of an inorganic insulating material such as a silicon oxide (SiO₂) or an organic insulating material such as an insulating polymer. The mask layer 20 may be formed to have a relatively thin thickness, for example, about 20 nm to 70 nm.

The neural multifunctional device 100 may include a plurality of nanotubes 30 each vertically arranged on the region of the two-dimensional nanomaterial layer 10 exposed around the plurality of holes H10 by the mask layer 20. Each of the plurality of nanotubes 30 may have a passage P10 for substance movement therein.

The plurality of nanotubes 30 may be formed of a metal oxide. For example, the plurality of nanotubes 30 may be formed of zinc oxide. In this case, the plurality of nanotubes 30 may be referred to as zinc oxide nanotubes. These nanotubes 30 may be formed according to a growth method on the region of the two-dimensional nanomaterial layer 10 exposed around the hole H10. However, the material of the nanotube 30 is not limited to zinc oxide and may vary depending on need. In some cases, the nanotubes 30 may be formed of a metal or a ceramic. In connection with the methods of producing these materials, the disclosure of the present inventor's Korean Patent No. 10-154769 may be referred, the disclosure of which is incorporated herein by reference in its entirety. For example, a growing method of the zinc oxide nanotubes is a method to grow vertically by controlling the directionality by using a zinc-containing precursor and an oxygen-containing gas as precursors. For example, a height and growth form of a nanostructure may be controlled by adjusting a pressure in a chamber for growing a zinc oxide-based nanotube, a flow rate of a zinc-containing precursor such as DEZn (diethylzine), and an oxygen flow rate. In addition, nanotubes with a height of as small as several tens of nm to several tens of μm may be grown by adjusting the process parameters.

In one embodiment, each of the plurality of nanotubes 30 may have an inner diameter ranging from about 220 nm to 2 um and an outer diameter ranging from about 330 nm to 2.5 μm. If the outer diameter of the nanotube 30 is too large, it may damage a nerve cell or make it difficult to select a single cell, and if the inner diameter of the nanotube 30 is too small, delivery of a substance (medical substance) through the passage P10 may not be easy. Accordingly, the nanotube 30 may preferably have an inner diameter in the range of about 220 nm to 2 μm and an outer diameter in the range of about 330 nm to 2.5 μm as described above. Meanwhile, the length of each of the plurality of nanotubes 30 may be approximately 600 nm to 12 μm. In this case, in the embodiments of the present application, the nanotubes 30 may be advantageous in properly performing various functions.

The neural multifunctional device 100 may further include a binding layer 40 arranged to fill a region between and around the plurality of nanotubes 30 on the mask layer 20 to a given height, and may further include an electrode structure 50 arranged to be electrically connected to at least a portion of the plurality of nanotubes 30 on the binding layer 40.

The binding layer 40 binds and supports the plurality of nanotubes 30 and may serve as a mold. The binding layer 40 may serve to support the device 100 as a whole. Accordingly, the binding layer 40 may be referred to as a support or a support layer. The binding layer 40 may be formed of a flexible material such as polymer (an insulating polymer). An upper surface of the binding layer 40 may be disposed to be somewhat recessed from the top of the nanotube 30. Accordingly, the plurality of nanotubes 30 may protrude upward to some extent with respect to a surface (upper surface) of the binding layer 40. For example, approximately 1/10 to ½ of the total length of the nanotube 30 may protrude above the binding layer 40. The protruding portion of the nanotube 30 may be inserted into or placed in contact with a nerve cell.

The electrode structure 50 may include at least one electrode pad 50a. In addition, although not shown, the electrode structure 50 may further include ‘an electrode wiring’ connecting the electrode pad 50a and the nanotube 30. A plurality of electrode pads 50a may be formed, and a plurality of the electrode wirings may be formed. The electrode structure 50 may include a type of ‘an electrode array structure’.

Furthermore, although not shown in FIG. 1, at least a portion of an outer peripheral surface of the nanotube 30 may be coated with a metal or a metallic substance. As a result, a coating layer surrounding at least a portion of the outer peripheral surface of the nanotube 30 may be further formed, and the coating layer may include a metal or a metallic substance. The coating layer may extend between the nanotube 30 and the binding layer 40.

In the neural multifunctional device 100 according to an embodiment of the present invention, the plurality of nanotubes 30 may be configured to perform at least one of electrical measurement and stimulation application to a nerve cell, and transfer substance to the nerve cell through the passage P10 therein. In one embodiment, the nanotube 30 may be in contact with a nerve cell and serve to measure the electrical potential of the nerve cell or apply electrical stimulation to the nerve cell. In addition, it may serve to deliver a substance (fluid containing a medicinal substance) to the nerve cell through the passage P10 therein. Therefore, it is possible to implement both of a function of a probe (or an electrode) capable of electrical measurement and a function of a channel for substance (fluid) movement by using the nanotube 30. The plurality of nanotubes 30 may be a ‘probe array’ for electrical measurement and, at the same time, may be a ‘channel array’ (multi-channel structure) for substance movement. From this perspective, the neural multifunctional device 100 may be said to have ‘multi-function’.

As the existing signal measurement devices for nerve cells use needle-type probes using nanowires, they have the limitation of only being able to measure signals. Therefore, in order to supply a nerve substance (medicinal substance) into a nerve cell during signal measurement using a signal measurement device, a separate nerve substance delivery device (passage) must be provided. However, in this case, it is not easy to place the nerve substance delivery device (passage) by avoiding the signal measurement device, but the accuracy of nerve substance transfer may also be reduced. Furthermore, because two devices must be handled, the measurement process may be complicated and difficult.

However, according to an embodiment of the present invention, as electrical signal measurement (or stimulation application) and substance transfer functions may be performed together by using a single neural multifunctional device 100, the effects that it is easy to make measurement and measuring accuracy may be improved may be obtained. In particular, since it is possible to easily measure the response (i.e., signal change) electrically while delivering a certain substance (medicinal substance) to a nerve cell, research and experiments on nerve cells may be facilitated, and evaluation of a certain medicinal substance may also be facilitated.

Furthermore, the neural multifunctional device 100 according to an embodiment of the present invention may be a flexible device. The neural multifunctional device 100 may be easily manufactured as a form of a flexible device due to the use of the two-dimensional nanomaterial layer 10. As the two-dimensional nanomaterial layer 10 is not only flexible itself but also may be easily transferred from one substrate to another substrate, it may be advantageous for implementing flexible devices. If the neural multifunctional device 100 is a flexible device, the usability of the device may be increased and the field of application may be expanded.

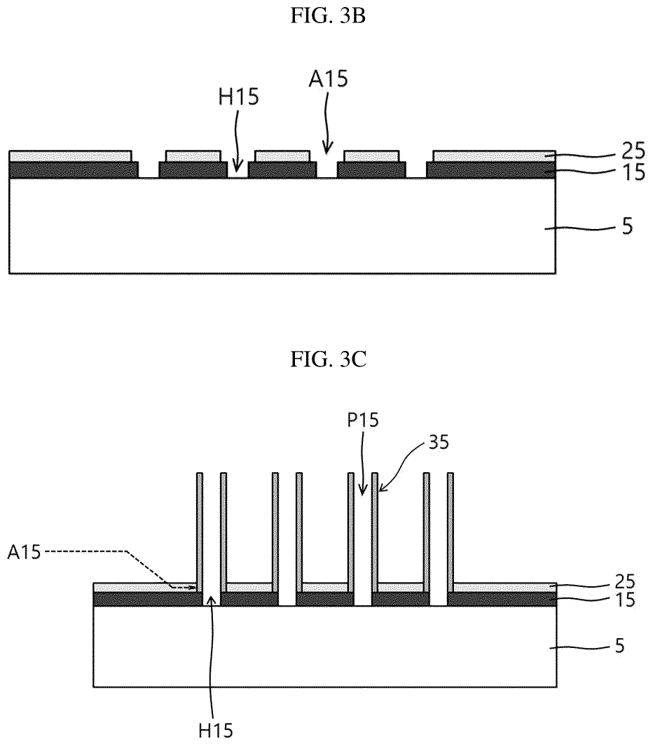

FIG. 2 is a schematic diagram illustrating a process for performing electrical measurement and substance transfer to a nerve cell C1 by using the neural multifunctional device 100 according to an embodiment of the present invention.

Referring to FIG. 2, the neural multifunctional device 100 according to an embodiment of the present invention may be placed on a predetermined pedestal S1, and the nerve cell C1 may be placed on the neural multifunctional device 100. For example, the pedestal S1 may be arranged to support an edge portion of the lower surface of the neural multifunctional device 100. The nerve cell C1 may be placed on the plurality of nanotubes 30 so as to contact them. The protruding portion of the nanotube 30 may contact or be inserted into the nerve cell C1.

While measuring the electrical signal of the nerve cell C1 by using the neural multifunctional device 100, a certain substance M1 may be delivered to the nerve cell C1 through the plurality of nanotubes 30. The substance M1 may be delivered to a specific microscopic region of the nerve cell C1. Here, the substance M1 may be a medicinal substance (or nerve substance) and may have a form of a fluid such as a solution. At this time, if necessary, the substance M1 may be supplied to a lower side of the neural multifunctional device 100 by using a predetermined supply tube 200. Supply of the substance M1 using the supply tube 200 may be performed very easily. When the substance M1 is supplied, the substance M1 may be delivered to the nerve cell C1 through the plurality of nanotubes 30. At this time, principles such as capillary pressure may be applied to the transfer of the substance M1.

If the neural multifunctional device 100 is used, measurements may be performed on a single neuron (i.e., a nerve cell) basis even in a narrow area. If necessary, simultaneous measurement for multiple neurons may be performed by increasing the size of the neural multifunctional device 100 or using a plurality of the neural multifunctional devices 100.

As such, according to an embodiment of the present invention, electrical measurement of the nerve cell C1 and substance transfer to the nerve cell C1 may be performed together by using a single neural multifunctional device 100. Since electrical measurement and substance transfer to the nerve cell C1 may be performed simultaneously by using one device 100, then effects that measurement becomes easier and measurement accuracy is improved may be obtained. In particular, since a certain substance may be delivered to the nerve cell C1 and the response (i.e., signal change) may be measured electrically in situ, research and experiments on the nerve cell Cl may be facilitated, and accurate evaluation (e.g., medicinal substance screening) of a given medicinal substance may also be facilitated.

In addition, according to an embodiment of the present invention, since the electrical signal of the nerve cell C1 may be measured in real time by using the nanotube 30, measurements may be made in units of several milliseconds over time or immediately after a specific stimulus is delivered to the nerve cell C1. In addition, medicinal substances using microfluidics may be delivered to the cultured nerve cell C1 (e.g., brain cell) for measurement of specific aspects or chemical control of the nerve cell C1 (e.g., brain cell) by using the passage of the nanotube 30.

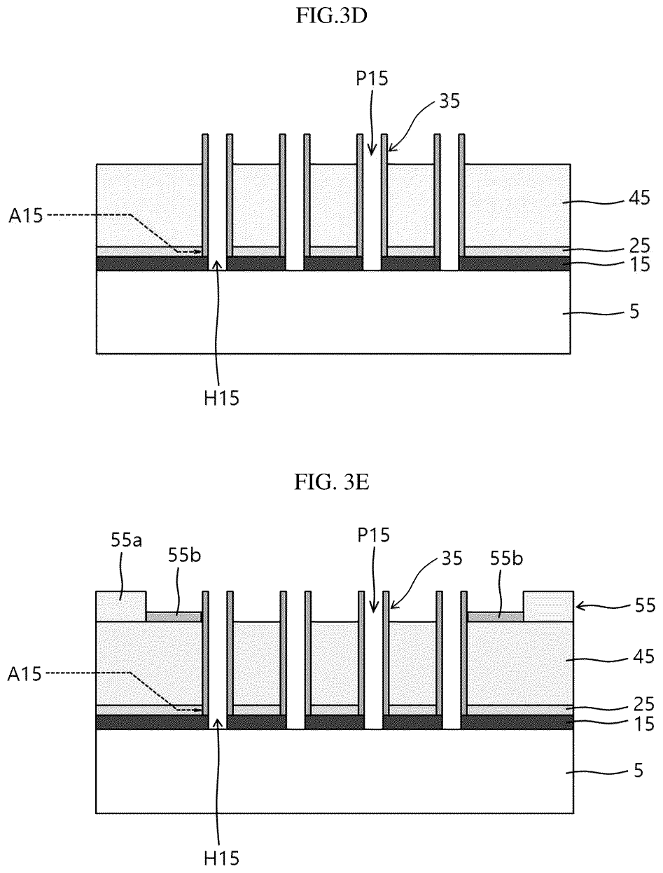

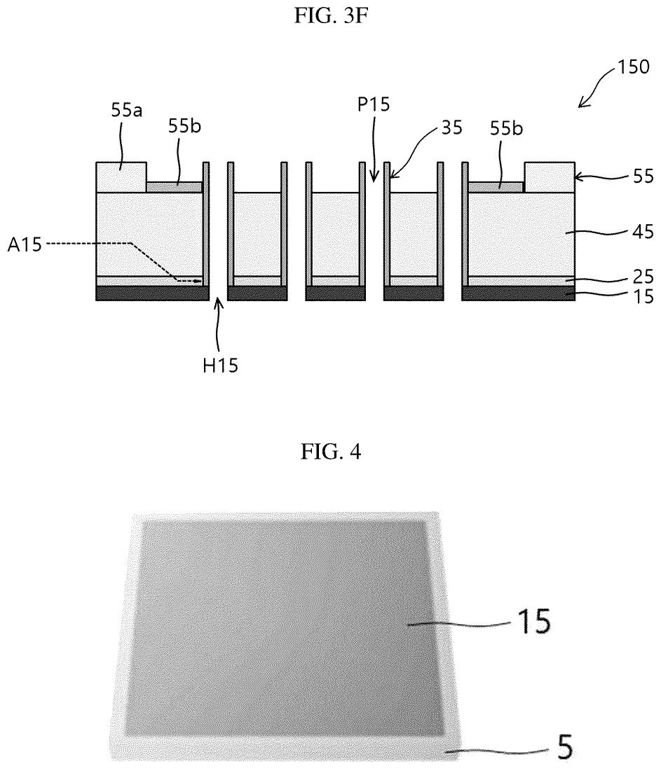

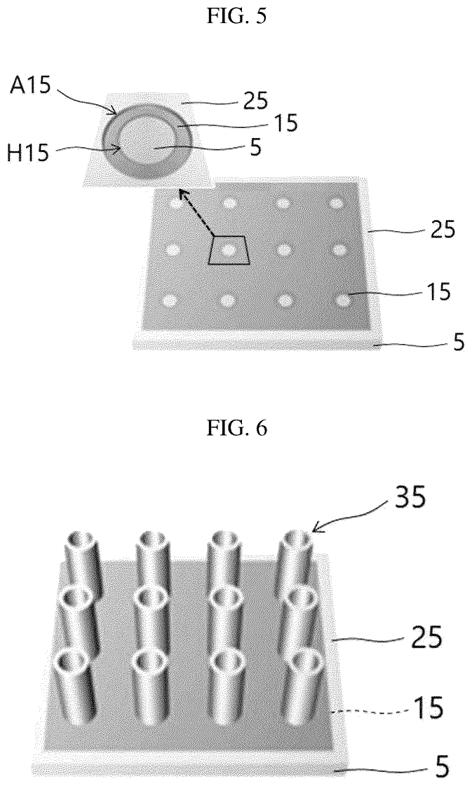

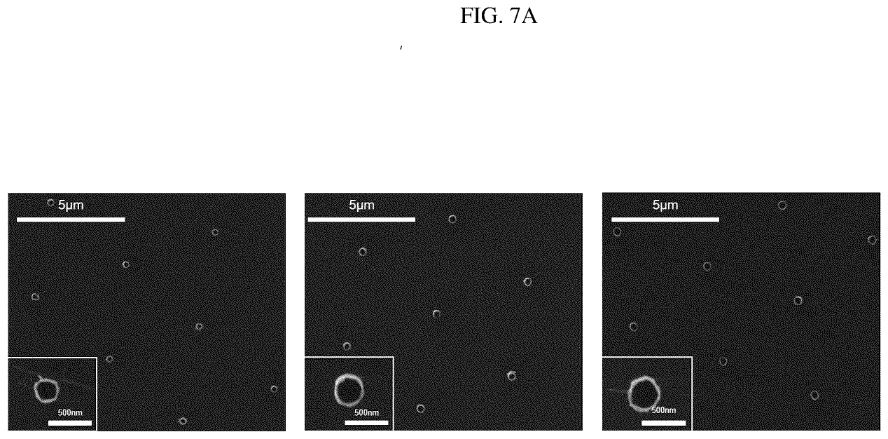

FIGS. 3A to 3F are cross-sectional diagrams illustrating a manufacturing method of a neural multifunctional device according to an embodiment of the present invention, FIG. 4 is a perspective diagram illustrating the result shown in FIG. 3A, FIG. 5 is a perspective diagram illustrating the result shown in FIG. 3B, and FIG. 6 is a perspective diagram illustrating the result shown in FIG. 3C.

Referring to FIG. 3A, a two-dimensional nanomaterial layer 15 may be disposed on a predetermined substrate 5. The two-dimensional nanomaterial layer 15 may be, for example, a graphene layer. The two-dimensional nanomaterial layer 15 may include a single-layer or a multi-layer graphene. The two-dimensional nanomaterial layer 15 may be transferred onto the substrate 5 or may be grown on the substrate 5. As a result, as shown in FIG. 4, a structure in which the two-dimensional nanomaterial layer 15 is disposed on the substrate 5 may be obtained.

Referring to FIG. 3B, a mask layer 25 may be formed on the two-dimensional nanomaterial layer 15. A plurality of holes H15 may be formed in the two-dimensional nanomaterial layer 15, and a plurality of openings A15 exposing each of the plurality of holes H15 of the two-dimensional nanomaterial layer 15 may be formed in the mask layer 25. The opening A15 may have a larger diameter than that of the hole H15 corresponding thereto. For example, the opening A15 and the corresponding hole H15 may have the same center, and the diameter of the opening A15 may be larger than the diameter of the hole H15. Accordingly, a region of the two-dimensional nanomaterial layer 15 around the hole H15 may be exposed by the opening A15. The region of the two-dimensional nanomaterial layer 15 exposed around the hole H15 may have a ring shape when observed from above.

First of all, the plurality of holes H15 may be formed in the two-dimensional nanomaterial layer 15, then a mask material layer may be formed thereon, and the plurality of openings A15 may be formed in the mask material layer. Thus, the mask layer 25 may be formed as shown in FIG. 3B. Alternatively, after forming a mask material layer on the two-dimensional nanomaterial layer 15 as shown in FIG. 3A, the plurality of openings A15 may be formed in the mask material layer, and the plurality of the hole H15 may be formed in the two-dimensional nanomaterial layer 15, and as a result, the structure as shown in FIG. 3B may be formed. In addition, the method for forming the structure shown in FIG. 3B may be modified in various ways.

The mask layer 25 may be formed of a certain insulating material. For example, the mask layer 25 may be formed of an inorganic insulating material such as silicon oxide (SiO₂) or an organic insulating material such as an insulating polymer. The mask layer 25 may be formed to have a relatively thin thickness, for example, about 20 nm to 70 nm. Accordingly, a result such as that shown in FIG. 5 may be formed. However, the structure of FIG. 5 is an example and may be modified depending on the case.

Referring to FIG. 3C, a plurality of nanotubes 35 may be formed on the region of the two-dimensional nanomaterial layer 15 exposed around the plurality of holes H15 by the mask layer 25. The plurality of nanotubes 35 may be formed through a growth method, for example, a selective growth method. Each of the plurality of nanotubes 35 may have a passage P15 for substance movement therein.

The plurality of nanotubes 35 may be formed of a metal oxide. For example, the plurality of nanotubes 35 may be formed of a zinc oxide (Zn oxide). In other words, the plurality of nanotubes 35 may be Zn oxide nanotubes. These nanotubes 35 may be easily formed through a growth method on the region of the two-dimensional nanomaterial layer 15 exposed around the hole H15. However, the material of the nanotubes 35 is not limited to zinc oxide and may vary as needed. In some cases, the nanotubes 35 may be formed of a metal or a ceramic.

Each of the plurality of nanotubes 35 may have an inner diameter ranging from about 220 nm to 2 μm and an outer diameter ranging from about 330 nm to 2.5 μm. If the outer diameter of the nanotube 35 is too large, it may cause damage to a nerve cell, and if the inner diameter of the nanotube 35 is too small, it may not be easy to transmit a substance (medicinal substance) through the passage P15. Accordingly, the nanotubes 35 may preferably have an inner diameter in the range of about 220 nm to 2 μm and an outer diameter in the range of about 330 nm to 2.5 μm as described above. Meanwhile, the length of each of the plurality of nanotubes 35 may be about 600 nm to 12 μm. In this case, in the embodiments of the present application, the nanotubes 35 may be advantageous in properly performing various functions.

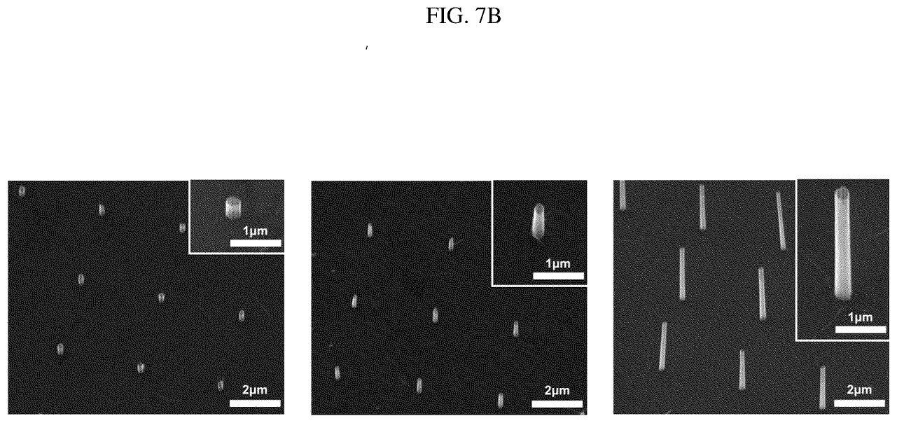

When the two-dimensional nanomaterial layer 15 is a graphene layer, since the plurality of nanotubes 35 may be easily formed on a large-area graphene layer by selective growth, the manufacturing process may be easy and the possibility of commercialization of the device may be high. Accordingly, a result such as that shown in FIG. 6 may be formed. FIG. 7A to FIG. 7C are electron microscope images of a plurality of nanotubes formed to have different inner diameters, outer diameters, and heights according to an embodiment of the present invention. Referring to FIG. 7A, it is possible to implement a plurality of nanotubes which have empty inside and have selected inner and outer diameters. Referring to FIG. 7B, in terms of the length of a plurality of nanotubes, the growth conditions may be changed to grow the plurality of nanotubes to have a designed length. Furthermore, referring to FIG. 7C, a plurality of nanotubes having a predetermined inner diameter, outer diameter, and height may be uniformly grown in an array form.

Referring again to FIG. 3D, a binding layer 45 arranged to fill a region between and around the plurality of nanotubes 35 to a given height may be formed on the mask layer 25. The binding layer 45 may be formed of a flexible material such as, for example, polymer (insulating polymer). The binding layer 45 may be formed by a coating method using a solution process, etc. An upper surface of the binding layer 45 may be disposed to be somewhat recessed from the top of the nanotube 35. Accordingly, the plurality of nanotubes 35 may protrude upward to some extent with respect to a surface (upper surface) of the binding layer 45. The protruding portion of the nanotube 35 may be inserted into or placed in contact with a nerve cell.

In another embodiment, first of all, an insulating layer to be the binding layer 45 is formed on the mask layer 25, through holes in which a plurality of nanotubes 35 will be formed are formed, and then a plurality of nanotubes 35 may be grown through the inside of the through holes. In this case, the binding layer 45 formed from the insulating layer may function as a mold layer which forms the plurality of nanotubes 35 and supports them at the same time.

Referring to FIG. 3E, an electrode structure 55 electrically connected to at least a portion of the plurality of nanotubes 35 may be formed on the binding layer 45. The electrode structure 55 may include at least one electrode pad 55a. Furthermore, the electrode structure 55 may further include an electrode wiring 55b which electrically connects the electrode pad 55a and the nanotube 35. A plurality of electrode pads 55a may be formed, and a plurality of electrode wirings 55b may also be formed. The electrode structure 55 may be said to include a type of ‘an electrode array structure’.

Thereafter, a device structure including the two-dimensional nanomaterial layer 15, the mask layer 25, the plurality of nanotubes 35, the binding layer 45, and the electrode structure 55 may be separated from the substrate 5. Since the two-dimensional nanomaterial layer 15 may be coupled to the substrate 5 by van der Waals force, the two-dimensional nanomaterial layer 15 may be easily separated from the substrate 5. The result is shown in FIG. 3F.

Referring to FIG. 3F, a neural multifunctional device 150 including the two-dimensional nanomaterial layer 15, the mask layer 25, the plurality of nanotubes 35, the binding layer 45, and the electrode structure 55 may be formed.

Depending on the case, the timing of separating the two-dimensional nanomaterial layer 15 from the substrate 5 may vary. For example, before forming the electrode structure 55, that is, in the step of FIG. 3D, a device structure including the two-dimensional nanomaterial layer 15, the mask layer 25, the plurality of nanotubes 35, and the binding layer 45 may be separated from the substrate 5. In this case, the neural multifunctional device 150 as shown in FIG. 3F may be formed by forming the electrode structure 55 after the separation step.

Furthermore, if necessary, the neural multifunctional device 150 as shown in FIG. 3F may be transferred to a separate substrate. At this time, the separate substrate may be a flexible substrate. The two-dimensional nanomaterial layer 15 has the advantage that it is easy to separate from the initial substrate, that is, the substrate 5 of FIG. 3E, and also easy to transfer to another substrate. Moreover, the two-dimensional nanomaterial layer 15 has the advantage that it may be transferred to an arbitrarily curved substrate surface.

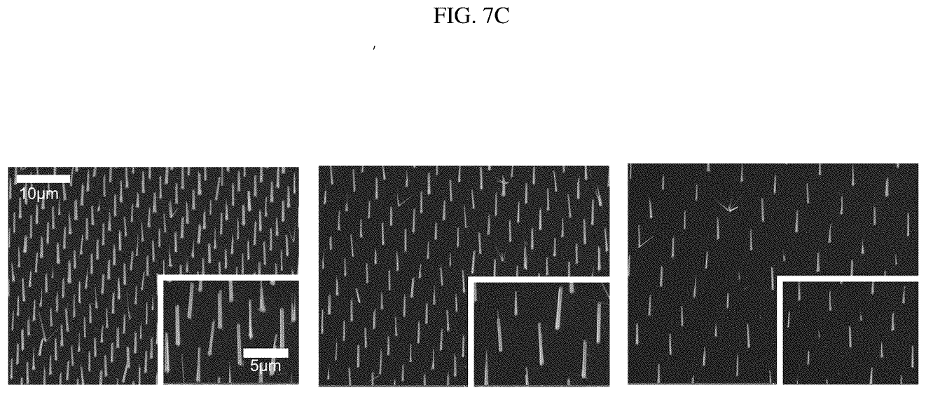

FIG. 8 is an electron microscope image of a portion of a neural multifunctional device manufactured according to an embodiment of the present invention. Referring to FIG. 8, it may be seen that the nanotube protrudes above the binding layer.

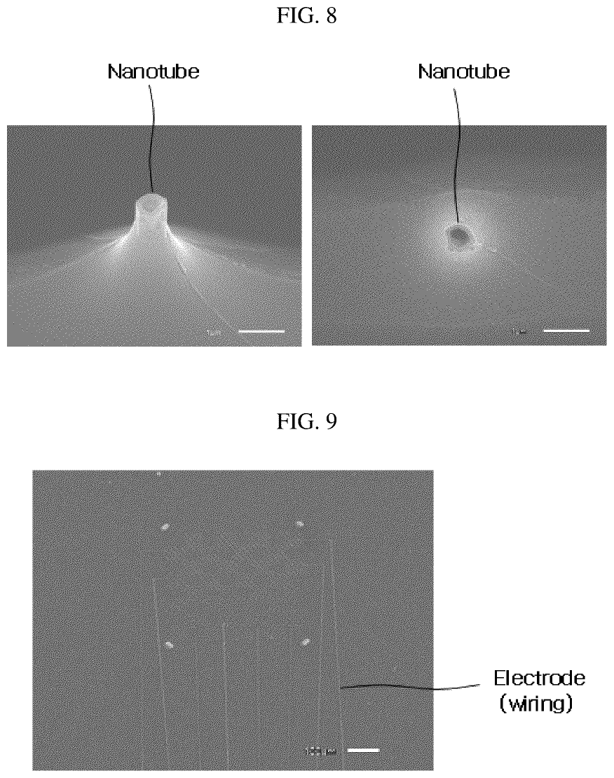

FIG. 9 is an electron microscope image taken from above of a portion of a neural multifunctional device manufactured according to an embodiment of the present invention. Referring to FIG. 9, a plurality of electrodes (wirings) connected to nanotubes may be seen.

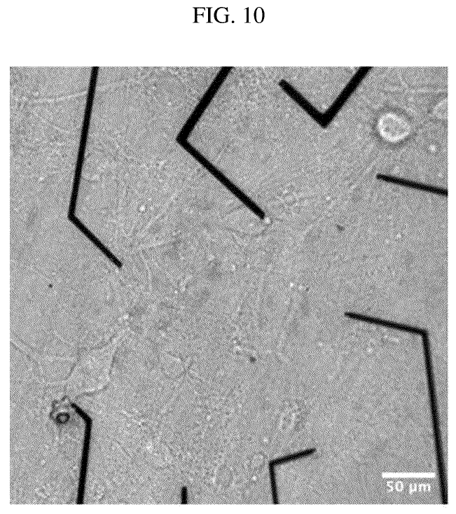

FIG. 10 is an electron microscope image illustrating a case where nerve cells were cultured on a neural multifunctional device manufactured according to an embodiment of the present invention. Referring to FIG. 10, it may be seen that nerve cells and neurites are entangled on the electrode array structure of the neural multifunctional device.

According to the embodiments of the present invention described above, it is possible to implement a neural multifunctional device which may perform at least one of electrical measurement and stimulation application to a nerve cell and also perform delivery of a substance (medicinal substance) to a nerve cell. A process for simultaneously performing substance transfer while performing electrical measurement/stimulation on a nerve cell by applying an appropriate electric signal through an electrode wiring structure by using a single device may be performed, or conversely, the electrical responses from a nerve cell may be measured by receiving an electrical signal. Furthermore, this process may be done in real time, and thus, measurement may be easier and measurement accuracy may be improved. In particular, the process for delivering a certain substance (medicinal substance) to a nerve cell and easily measuring the response thereto in an electrical manner not only makes it easy to conduct research/experiments on the nerve cell, but also allows for evaluation of a certain medicinal substance to become easier.

In addition, according to embodiments of the present invention, since it is possible to implement a neural multifunctional device which may be easily manufactured as a form of a flexible device by using a two-dimensional nanomaterial, it may be advantageous in expanding the utilization and application fields of the neural multifunctional device.

A neural multifunctional device according to embodiments of the present invention may also be referred to as ‘a neural network device.’ The neural multifunctional device may be a flexible multi-channel device which may simultaneously measure intracellular signals on a single cell basis and deliver medicinal substances, and may be used for medicinal substance screening, development of therapeutics, measurement of nerve cell networks using an array, development of medical devices, and implementation of artificial neural tissue, and the like. In addition, it may be applied complexly to various fields of biomedical engineering devices which require delivery of medicinal substances and measurement of electrical signals (or application of stimulation).

In this specification, the preferred embodiments of the present invention have been disclosed, and although specific terms have been used, they are only used in a general sense to easily explain the technological content of the present invention and to help understanding the present invention, and they are not used to limit the scope of the present invention. It is obvious to those having ordinary skill in the related art to which the present invention belong that other modifications based on the technological idea of the present invention may be implemented in addition to the embodiments disclosed herein. It will be understood to those having ordinary skill in the related art that in connection with neural multifunctional devices and methods of manufacturing the same according to the embodiments described with reference to FIGS. 1 to 10, various substitutions, changes, and modifications may be made without departing from the technological spirit of the present invention. Therefore, the scope of the invention should not be determined by the described embodiments, but should be determined by the technological concepts described in the claims.

1. A neural multifunctional device comprising:
a two-dimensional nanomaterial layer in which a plurality of holes are formed;
a mask layer disposed on the two-dimensional nanomaterial layer and having a plurality of openings each exposing the plurality of holes, wherein the opening has a larger diameter than that of the hole corresponding thereto and is formed to expose a region of the two-dimensional nanomaterial layer around the hole;
a plurality of nanotubes each vertically disposed on the region of the two-dimensional nanomaterial layer exposed around the plurality of holes by the mask layer and having a passage for substance movement therein;
a binding layer disposed on the mask layer to fill a region between and around the plurality of nanotubes to a given height; and
an electrode structure disposed on the binding layer to be electrically connected to at least a portion of the plurality of nanotubes,
wherein the plurality of nanotubes are configured to perform at least one of electrical measurement and stimulation application to a nerve cell and transfer of substance to the nerve cell through the passage.

2. The neural multifunctional device of claim 1, wherein the two-dimensional nanomaterial layer includes graphene.

3. The neural multifunctional device of claim 1, wherein the plurality of nanotubes are formed of a metal oxide.

4. The neural multifunctional device of claim 3, wherein the plurality of nanotubes are formed of a Zn oxide.

5. The neural multifunctional device of claim 1, wherein each of the plurality of nanotubes has an inner diameter ranging from 220 nm to 2 μm and an outer diameter ranging from 330 nm to 2.5 μm.

6. The neural multifunctional device of claim 1, wherein each of the plurality of nanotubes may have a length ranging from 600 nm to 12 μm.

7. The neural multifunctional device of claim 1, wherein the plurality of nanotubes have a protruding shape with respect to a surface of the binding layer.

8. The neural multifunctional device of claim 1, wherein the binding layer includes polymer.

9. The neural multifunctional device of claim 1, wherein the neural multifunctional device is a flexible device.

10. A manufacturing method of a neural multifunctional device comprising:
forming a stack in which a two-dimensional nanomaterial layer and a mask layer are sequentially arranged on a substrate, wherein the two-dimensional nanomaterial layer has a plurality of holes, the mask layer has a plurality of openings each exposing the plurality of holes, and the opening has a larger diameter than that of the hole corresponding thereto and exposes a region of the two-dimensional nanomaterial layer around the hole;
forming a plurality of nanotubes each vertically disposed on the region of the two-dimensional nanomaterial layer exposed around the plurality of holes by the mask layer and having a passage for substance movement therein;
forming a binding layer on the mask layer to fill a region between and around the plurality of nanotubes to a given height; and
forming an electrode structure arranged to be electrically connected to at least a portion of the plurality of nanotubes on the binding layer,
wherein the plurality of nanotubes are configured to perform at least one of electrical measurement and stimulation application to a nerve cell and transfer of substance to the nerve cell through the passage.

11. The manufacturing method of a neural multifunctional device of claim 10, further comprising separating a device structure including at least the two-dimensional nanomaterial layer, the mask layer, the plurality of nanotubes, and the binding layer from the substrate after the forming the binding layer or the forming the electrode structure.

12. The manufacturing method of a neural multifunctional device of claim 10, wherein the two-dimensional nanomaterial layer includes graphene.

13. The manufacturing method of a neural multifunctional device of claim 10, wherein the plurality of nanotubes are formed of a metal oxide.

14. The manufacturing method of a neural multifunctional device of claim 13, wherein the plurality of nanotubes are formed of a Zn oxide.

15. The manufacturing method of a neural multifunctional device of claim 10, wherein the plurality of nanotubes have a protruding shape with respect to a surface of the binding layer.

16. The manufacturing method of a neural multifunctional device of claim 10, wherein the binding layer includes polymer.

17. A neural multifunctional device comprising:
a two-dimensional nanomaterial layer in which a plurality of holes are formed;
a mask layer disposed on the two-dimensional nanomaterial layer and having a plurality of openings each exposing the plurality of holes;
a plurality of nanotubes each vertically disposed on the region of the two-dimensional nanomaterial layer exposed around the plurality of holes by the mask layer;
a binding layer disposed on the mask layer to fill a region between and around the plurality of nanotubes to a given height; and
an electrode structure disposed on the binding layer to be electrically connected to at least a portion of the plurality of nanotubes.

18. The neural multifunctional device of claim 17, wherein the two-dimensional nanomaterial layer includes graphene.

19. The neural multifunctional device of claim 17, wherein each of the plurality of nanotubes may have a length ranging from 600 nm to 12 μm.

20. The neural multifunctional device of claim 17, wherein the plurality of nanotubes have a protruding shape with respect to a surface of the binding layer.
